(19) United States
(12) Patent Application Publication
(10) Pub. No.: US 2026/0334758 A1
(43) Pub. Date: Oct. 29, 2026

(54) ELECTRONICS MODULE FOR INTEGRATION INTO A HANDGRIP OF A MEDICAL INSTRUMENT, HANDGRIP AND MEDICAL INSTRUMENT

(71) Applicant: OLYMPUS Winter & Ibe GmbH, Hamburg (DE)

(72) Inventors: Sebastian Jungbauer, Hamburg (DE); Andreas Mueckner, Hamburg (DE)

(73) Assignee: OLYMPUS Winter & Ibe GmbH, Hamburg (DE)

(21) Appl. No.: 19/381,341

(22) Filed: Nov. 6, 2025

(30) Foreign Application Priority Data
Nov. 6, 2024 (EP) 24 211 036.9

(51) Int. Cl. A61B 1/00; A61B 90/00

(52) U.S. Cl. CPC A61B 1/00066; A61B 1/00016; A61B 90/06; A61B 2090/064; A61B 2560/0214

(57) ABSTRACT

An electronics module for integration into a handgrip of a medical instrument. The electronics module includes a printed circuit board (PCB), including a first part of the PCB and a second part of the PCB, the first part of the PCB being at least as wide as a second part of the PCB. Wherein the first and second parts are arranged such that the second part is positioned in a first plane different from a second plane of the first part, when viewed in a top view onto the first part, the second part at least concerning a width direction is contained within a width of the first part, and the first and second parts are interconnected. The electronics module further includes electronic components provided to the PCB; and an encapsulation material encapsulating the PCB, wherein the encapsulation material is resilient to sterilization processes.

### CROSS-REFERENCE TO RELATED APPLICATION

The present application is based upon and claims the benefit of priority from EP 24 211 036.9 on Nov. 6, 2024, the entire contents of which is incorporated herein by reference.

### BACKGROUND

### Field

The present disclosure relates to an electronics module, in particular a sensor data logger, for integration into a handgrip of a medical instrument, a handgrip of a medical instrument comprising such an electronics module, and a medical instrument, comprising such a handgrip.

### Prior Art

Recently, electronics modules have been developed, which are to be integrated into handgrips of medical instruments, such as for example sensor data logger devices, which are adapted to record usage data of the corresponding medical devices and to store and/or transmit said data for further processing. One particular example for such a device concerns the integration of accelerometers into respective handgrips, which can detect drops and impacts on medical instruments, in order to monitor their usage and predict possible damage thereof. However, other types of sensors may also be employed to ensure that the corresponding device has not been subjected to potentially harmful conditions.

Electronics modules of these types are capable of standalone operation, such that they can perform their functionality even if the handgrip itself is not mechanically and electrically connected to a superordinate structure of the medical instrument, which requires the integration of an independent power source. It also has to be kept in mind that over their lifetime, such handgrips regularly have to be subjected to sterilization processes, such as steam sterilization, plasma sterilization or treatment in an automated washer disinfector, which might damage electronic components if they are not protected in a suitable manner.

### SUMMARY

There is therefore the need for electronics modules for integration into handgrips of medical instruments, which on the one hand fulfil the requirements on size and geometry to be able to be integrated into the small inner spaces of such handgrips, while on the other hand it is necessary that the electronics modules will not be impaired or damaged during sterilization processes of the handgrips themselves.

It is therefore an object to provide such an electronics module which has geometrical dimensions that make it suitable for integration into the handgrip of a medical instrument where it can perform its intended functionality and at the same time is resistant to frequent sterilization processes which are performed on the handgrip over its lifespan.

For this purpose, an electronics module is provided, for example, a sensor data logger, for integration into a handgrip of a medical instrument, comprising a two-part printed circuit board, PCB, wherein a first part of the PCB is at least as wide as the second part of the PCB, the two parts are arranged in such a manner that the second part is positioned in a plane different from the plane of the first part, in a top view onto the first part the second part at least concerning a width direction is contained within the width of the first part and the two parts are interconnected, wherein the electronics module further comprises electronic components provided to the PCB, and an encapsulation material encapsulating the PCB, wherein the encapsulation material is resilient to sterilization processes.

By thus distributing the PCB with its electronic components onto two different planes, which may for example be stacked on top of one another with respect to the width direction of the first part, and by furthermore encapsulating the entire PCB into a suitable encapsulation material, both a compact design thereof and a high resiliency to external influences can be achieved, and additionally, sterilization processes can be achieved.

In an embodiment, selected electronic components may project out of the encapsulation material, such as for example antennas for a communication with external communication devices, which may extend beyond the PCBs and are as such not as sensitive to sterilization processes as other electronics components.

The module may further comprise at least one energy storage, such as a battery, for example a primary coin cell, for powering the electronic components. In different embodiments, other types of energy storage may also be provided, such as for example rechargeable batteries and/or supercapacitors. By providing such energy storage, no electrical coupling between the PCB of the electronics module and an external power source or the main medical instrument is required.

Independent of the type of energy storage employed in the electronics module, said at least one energy storage may also be encapsulated by the encapsulation material, such that in the case of rechargeable energy storage, a charging interface can extend out of the encapsulation material or the capability for contactless charging can be provided.

While different encapsulation materials are known in the art, which are resilient to sterilization processes, such as steam sterilization, plasma sterilization and treatments in automated washer disinfectors, the encapsulation material of the module may for example be a silicone compound or a thermosetting polymer compound, which are both relatively cheap and easy to apply on the respective components to be protected. Such encapsulation materials can be applied in low-temperature processes, such that the electronic components as well as possible solder connections and energy storage are not deteriorated.

In order to be able to not only achieve a standalone operation of the electronics module, but to also be able to transmit data from the electronic components to an external communication device, the electronic components may comprise an antenna system and a radio function for wireless communication with said external communication device integrated in the electronic components. For this purpose, known communication standards and frequencies may be used, or a proprietary protocol may be employed, and different transmission frequencies with corresponding dimensions of the antenna system may be chosen, depending on the concrete implementation of the data transfer.

As already briefly mentioned above, the electronic components may be adapted for a regular collection of sensor data by at least one sensor, which can be comprised therein, for example concerning temperature, pressure, humidity, acceleration, bending forces, mechanical forces and/or the presence of chemical substances. By providing such a sensor, usage conditions of the corresponding medical instrument may be logged over time in order to ensure that said instrument has not been subjected to irregular conditions, which might cause damage thereto.

Concerning the arrangement of the first and second parts of the PCB, the plane of the first part of the PCB may be parallel to the plane of the second part of the PCB, however, different angles between the two parts are also conceivable, depending on the geometrical constraints given on the electronics module at its mounting position within the corresponding handgrip.

While the first part and the second part of the PCB may furthermore be formed as two components of a single flexible or bent PCB, they may also be embodied by separate boards interconnected at a longitudinal end region thereof, such as by a flexible PCB connection. However, other types of connections between the two parts of the PCB may also be implemented, such as wiring in a longitudinally central part thereof.

According to a further aspect, a handgrip of a medical instrument is provided, comprising an outer shell delimiting an inner space of the handgrip, and an electronics module, as discussed above, positioned in the inner space of the handgrip, such as positioned next to the inner wall of the outer shell. Additionally, a partitioning wall inside the inner space of the handgrip may be provided, which together with the outer shell defines a compartment of the inner space, in which the electronics module is housed. Additional partition walls at the longitudinal ends of the compartment may additionally be provided, in order to further encapsulate the electronics module on all sides thereof, wherein at least one of the partition walls may be opened and closed for improved accessibility to the electronics module.

The encapsulation material may substantially fill the entire compartment for at least a longitudinal section of the inner space of the handgrip and/or the partitioning wall may be positioned inside the inner space of the handgrip in such a manner that in a cross-section view, the compartment accounts for less than half of the cross-section area of the inner space.

Additionally or alternatively, the outer shell may have a substantially circular cross-section, such that the electronics module, if positioned at a radially outer position adjacent to the inner wall of said outer shell, may have an encapsulation in the form of a cylinder cut-out.

Furthermore, a medical instrument is provided, such as a video laparoscope, comprising a handgrip as described above.

### BRIEF DESCRIPTION OF THE DRAWINGS

Further features and advantages will become even clearer from the following description of an embodiment thereof, when viewed together with the accompanying drawings. In the drawings:

FIG. 1 illustrates a cross-section view of a handgrip of a medical instrument comprising an electronics module in its inner space, and

FIG. 2 illustrates a side view of the electronics module of FIG. 1.

FIG. 3 illustrates a medical instrument having the handgrip of FIG. 1.

### DETAILED DESCRIPTION

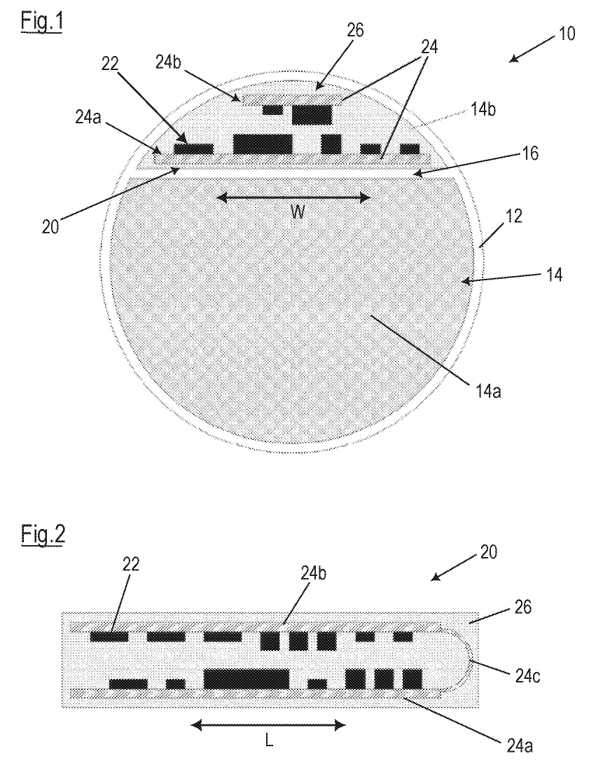

FIG. 1 shows a handgrip 10 of a medical instrument which may be gripped by a user, wherein the medical instrument may for example be a video laparoscope, in which typically light guides and further video components as well as mechanical components pass through the handgrip 10.

For this purpose, the handgrip 10 comprises an outer shell 12, which may for example be made of a plastic material and which delimits an inner space 14 of the handgrip 10. Said inner space 14 is divided into a main part 14a and a compartment 14b by a partitioning wall 16, wherein in the cross-section view of FIG. 1 it can be seen that the cross-section area taken up by the compartment 14b is smaller than the remaining free space of the main part 14a, in which for example light guides and video connections as well as mechanical components may be installed. Furthermore, a longitudinal direction and a width direction of the handgrip 10 are represented in FIGS. 1 and 2 by arrows L and W, respectively.

In the compartment 14b, which may also be further delimited at its longitudinal ends in the longitudinal direction L of the handgrip 10, an electronics module is housed, which is generally denoted with reference numeral 20 and again shown in FIG. 2 in a schematic side view.

Said electronics module 20 may for example be or comprise a sensor data logger, in turn comprising electronic components such as sensors, a controller comprising hardware, such as a processor, CPU or one or more circuits, an antenna system and battery cells, which are commonly referred to with reference number 22 and which are provided to a two-part printed circuit board, PCB, 24. In the embodiment shown in FIGS. 1 and 2, said two-part PCB comprises a wide first part 24a and a narrow second part 24b with respect to the width direction W, which extend in parallel planes and which are fitted to the available space in the compartment 14b inside the handgrip 10. Herein, in a top view onto the first part 24a, in FIG. 1 corresponding to a radial direction towards the center of the circular outer shell 12, the second part 24b is fully contained within the first part 24a, both concerning the longitudinal direction L and the width direction W.

In one of their longitudinal end regions, the first and second parts 24a, 24b of the PCB 24 are connected by a flexible PCB connection 24c, whereas in alternative variants, a flexible PCB bent into a shape with two corresponding parts 24a and 24b may also be used instead.

Furthermore, the PCB 24 and the electronic components 22 in their entirety are encapsulated within an encapsulation material 26 which fills the entire compartment 14b in at least part of the longitudinal direction L of the handgrip 10, in which the PCB 24 is present. By employing an encapsulation material for the encapsulation, which is resilient to sterilization processes to which the handgrip is expected to be subjected during its regular usage cycles, it can be ensured that the electronics module is unaffected by such treatments and that its operation will not negatively be affected by repeated sterilization processes to which the handgrip is subjected.

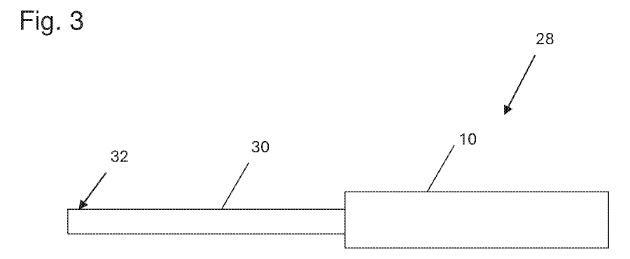

FIG. 3 illustrates a medical instrument 28 having the handgrip 10 illustrated in FIGS. 1 and 2 as well as an insertion portion 30, which can be rigid or flexible, and may have an articulating distal end. A distal end 32 of the insertion section 30 may have one or more components comprising an image sensor, illumination means and end-effector. In the medical instrument 28, the light guides, the video connections as well as the mechanical components for operating such components may be installed in one or more of the main part 14a of the handgrip 10 and in the insertion portion 30.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. An electronics module for integration into a handgrip of a medical instrument, the electronics module comprising:
a printed circuit board (PCB), comprising a first part of the PCB and a second part of the PCB, the first part of the PCB being at least as wide as a second part of the PCB,
wherein the first and second parts are arranged such that the second part is positioned in a first plane different from a second plane of the first part,
when viewed in a top view onto the first part, the second part at least concerning a width direction is contained within a width of the first part, and
the first and second parts are interconnected,
electronic components provided to the PCB; and
an encapsulation material encapsulating the PCB, wherein the encapsulation material is resilient to sterilization processes.

2. The electronics module according to claim 1, further comprising at least one energy storage for powering the electronic components.

3. The electronics module according to claim 2, wherein the at least one energy storage comprises one of a battery or a coin cell.

4. The electronics module according to claim 2, wherein the at least one energy storage is also encapsulated by the encapsulation material.

5. The electronics module according to claim 1, wherein the encapsulation material is one of a silicone compound or a thermosetting polymer compound.

6. The electronics module according to claim 4, wherein the encapsulation material is one of a silicone compound or a thermosetting polymer compound.

7. The electronics module according to claim 1, wherein the encapsulation material is applied in a low-temperature process.

8. The electronics module according to claim 1, wherein the electronic components comprise an antenna system and a radio function for wireless communication with an external communication device.

9. The electronics module according to claim 1, wherein the electronic components are configured for collection of sensor data by a sensor.

10. The electronics module according to claim 1, wherein the sensor data is one or more of temperature data, pressure data, humidity data, acceleration data, bending forces data, mechanical forces data and data representing the presence of chemical substances.

11. The electronics module according to claim 1, wherein the first plane of the first part of the PCB is parallel to the second plane of the second part of the PCB.

12. The electronics module according to claim 1, wherein the first part of the PCB and the second part of the PCB are interconnected by a third part at a longitudinal end region thereof.

13. The electronics module according to claim 1, wherein the third part comprises a flexible PCB connection.

14. A handgrip of a medical instrument, comprising:
an outer shell delimiting an inner space; and
the electronics module according to claim 1 positioned in the inner space of the outer shell.

15. The handgrip according to claim 14, further comprising a partitioning wall inside the inner space of the outer shell, which together with the outer shell defines a compartment of the inner space, in which the electronics module is housed.

16. The handgrip according to claim 15, wherein the encapsulation material substantially fills the entire compartment for at least a longitudinal section of the inner space of the outer shell.

17. The handgrip according to claim 14, wherein the partitioning wall is positioned inside the inner space of the outer shell such that in a cross-section view, the compartment accounts for less than half of the cross-sectional area of the inner space.

18. The handgrip according to claim 14, wherein the outer shell has a substantially circular cross-section.

19. A medical instrument comprising the handgrip according to claim 14.
